SEQUENCE LISTING (38 sequences)

SEQ ID NO: 1 - DNA, 1201 bp, Homo sapiens
agtcgccgctgggcaccccgagaccatggggaagctcgtggcgctggtcctgctgggggtcggcctgtccttagtcggggagatgttcctggcgtttagagaaagggtgaatgcctctcgagaagtggagccagtagaacctgaaaactgccaccttattgaggaacttgaaagtggctctgaagatattgatatacttcctagtgggctggcttttatctccagtggattaaaatatccaggcatgccaaactttgcgccagatgaaccaggaaaaatcttcttgatggatctgaatgaacaaaacccaagggcacaagcgctagaaatcagtggtggatttgacaaagaattatttaatccacatgggatcagtattttcatcgacaaagacaatactgtgtatctttatgttgtgaatcatccccacatgaagtccactgtggagatatttaaatttgaggaacaacaacgttctctggtatacctgaaaactataaaacatgaacttctcaaaagtgtgaatgacattgtggttcttggaccagaacagttctatgccaccagagaccactattttaccaactccctcctgtcattttttgagatgatcttggatcttcgctggacttatgttcttttctacagcccaagggaggttaaagtggtggccaaaggattttgtagtgccaatgggatcacagtctcagcagaccagaagtatgtctatgtagctgatgtagcagctaagaacattcacataatggaaaaacatgataactgggatttaactcaactgaaggtgatacagttgggcaccttagtggataacctgactgtcgatcctgccacaggagacattttggcaggatgccatcctaatcctatgaagctactgaactataaccctgaggaccctccaggatcagaagtacttcgcatccagaatgttttgtctgagaagcccagggtgagcaccgtgtatgccaacaatggctctgtgcttcagggcacctctgtggcttctgtgtaccatgggaaaattctcataggcaccgtatttcacaaaactctgtactgtgagctctagactctagatagtaaaaaaaaaaaaaaaaagtctacatattttgtaaaagtaaactgataattgtatgataagtggcactgtaagtaaatagcaaacaccaaaaaaaaaaaa

SEQ ID NO: 2 - protein, 354 aa, Homo sapiens
MGKLVALVLLGVGLSLVGEMFLAFRERVNASREVEPVEPENCHLIEELESGSEDIDILPSGLAFISSGLKYPGMPNFAPDEPGKIFLMDLNEQNPRAQALEISGGFDKELFNPHGISIFIDKDNTVYLYVVNHPHMKSTVEIFKFEEQQRSLVYLKTIKHELLKSVNDIVVLGPEQFYATRDHYFTNSLLSFFEMILDLRWTYVLFYSPREVKVVAKGFCSANGITVSADQKYVYVADVAAKNIHIMEKHDNWDLTQLKVIQLGTLVDNLTVDPATGDILAGCHPNPMKLLNYNPEDPPGSEVLRIQNVLSEKPRVSTVYANNGSVLQGTSVASVYHGKILIGTVFHKTLYCEL

SEQ ID NO: 3 - DNA, 1203 bp, Homo sapiens
atggggaagctcgtggcgctggtcctgctgggggtcggcctgtccttagtcggggagatgtttctggcgtttagagaaagggtgaatgcctctcgagaagtggagccagtagaacctgaaaactgccaccttattgaggaacttgaaagtggctctgaagatattgatatacttcctagtgggctggcttttatctccagtgtctgttggagtttgctggaagtccactccagaccctgtttgcctgggtatcaccagtggaggctgcagaacggcaaatattgctgcctgatttttcttctggaagcttcatcccagaggggcatccgcctgtatgagggattaaaatatccaggcatgccaaactttgcgccagatgaaccaggaaaaatcttcttgatggatctgaatgaacaaaacccaagggcacaagcactagaaatcagtggtggatttgacaaagaattatttaatccacatgggatcagtattttcatcgacaaagacaatactgtgtatctttatgttgtgaatcatccccacatgaagtccactgtggagatatttaaatttgaggaacaacaacgttctctggtatacctgaaaactataaaacatgaacttctcaaaagtgtgaatgacattgtggttcttggaccagaacagttctatgccaccagagaccactattttaccaactccctcctgtcattttttgagatgatcttggatcttcgctggacttatgttcttttctacagcccaagggaggttaaagtggtggccaaaggattttgtagtgccaatgggatcacagtctcagcagaccagaagtatgtctatgtagctgatgtagcagctaagaacattcacataatggaaaaacatgataactgggatttaactcaactgaaggtgatacagttgggcaccttagtggataacctgactgtcgatcctgccacaggagacattttggcaggatgccatcctaatcctatgaagctactgaactataaccctgaggaccctccaggatcagaagtacttcgcatccagaatgttttgtctgagaagcccagggtgagcaccgtgtatgccaacaatggctctgtgcttcagggcacctctgtggcttctgtgtaccatgggaaaattctcataggcaccgtatttcacaaaactctgtactgtgagctctag

SEQ ID NO: 4 - protein, 400 aa, Homo sapiens
MGKLVALVLLGVGLSLVGEMFLAFRERVNASREVEPVEPENCHLIEELESGSEDIDILPSGLAFISSVCWSLLEVHSRPCLPGYHQWRLQNGKYCCLIFLLEASSQRGIRLYEGLKYPGMPNFAPDEPGKIFLMDLNEQNPRAQALEISGGFDKELFNPHGISIFIDKDNTVYLYVVNHPHMKSTVEIFKFEEQQRSLVYLKTIKHELLKSVNDIVVLGPEQFYATRDHYFTNSLLSFFEMILDLRWTYVLFYSPREVKVVAKGFCSANGITVSADQKYVYVADVAAKNIHIMEKHDNWDLTQLKVIQLGTLVDNLTVDPATGDILAGCHPNPMKLLNYNPEDPPGSEVLRIQNVLSEKPRVSTVYANNGSVLQGTSVASVYHGKILIGTVFHKTLYCEL

SEQ ID NO: 5 - DNA, 655 bp, Homo sapiens
aacttgaaacagaatgtgtattatccttggttgtgtttccttggccctgcagcaggatgaagctctcctctggcatcattttctgctccctggtcctgggtgtcagcagccaaggatggttaacattcctcaaggcagctggccaagggactaaagacatgtggaaagcctactctgacatgaaagaagccaattacaaaaaattcagacaaatacttccatgcttgggggaactatgatgctgtacaaagggggcttggggctgtctgggctacagaagtgatcaggtaatgcacattcctgatgttgccaggaatgagtgagcagagcttgactgccttggacagtcaggagagagcgatgccagagagaacgtccagagactcacaggagaccatgcagaggattcgctggctggccaggctaccaacaaatggggccagagtggcaaagaccccaatcacttccgacctgctggcctgccagagaaatactgagcttccttttcaatctgctctcaggagacctggctgtgagcccctgagggcagggacatttgttgacctacagttactgaattctatatccctagtacttgatatagaacacataaaaatgcttaataaatgcttgtgaaatccaaaaaaaaaaaaaa

SEQ ID NO: 6 - protein, 60 aa, Homo sapiens [sequence omitted]

SEQ ID NO: 7 - protein, 10 aa, Artificial sequence
  Synthetic sequence Purification tag
MRGSHHHHHH

SEQ ID NO: 8 - protein, 8 aa, Artificial sequence
  Synthetic sequence Purification tag
DYKDDDDK

SEQ ID NO: 9 - protein, 15 aa, Artificial sequence
  Synthetic sequence Purification tag
KETAAAKFERQHMDS

SEQ ID NO: 10 - protein, 8 aa, Artificial sequence
  Synthetic sequence Purification tag
WSHPQFEK

SEQ ID NO: 11 - protein, 10 aa, Artificial sequence
  Synthetic sequence Purification tag
EQKLISEEDL

SEQ ID NO: 12 - protein, 12 aa, Artificial sequence
  Synthetic sequence Purification tag
MKAEFRRQESDR

SEQ ID NO: 13 - protein, 12 aa, Artificial sequence
  Synthetic sequence Purification tag
MRDALDRLDRLA

SEQ ID NO: 14 - DNA, 21 bp, Artificial sequence
  Synthetic sequence Forward primer
cctagtgggctggctttcatc

SEQ ID NO: 15 - DNA, 23 bp, Artificial sequence
  Synthetic sequence Reverse primer
ctggcttatctggtgcaaagtct

SEQ ID NO: 16 - DNA, 19 bp, Artificial sequence
  Synthetic sequence Probe
tccagtggattaaaatatc

SEQ ID NO: 17 - DNA, 20 bp, Artificial sequence
  Synthetic sequence Forward primer
gctacactgaggaccaggtt

SEQ ID NO: 18 - DNA, 24 bp, Artificial sequence
  Synthetic sequence Reverse primer
agcatcgaaggtagaagagtgagt

SEQ ID NO: 19 - DNA, 16 bp, Artificial sequence
  Synthetic sequence Probe
ctcctgcgacttcaac

SEQ ID NO: 20 - DNA, 26 bp, Artificial sequence
  Synthetic sequence Forward primer
ggttcctgctttcacagaataattcc

SEQ ID NO: 21 - DNA, 24 bp, Artificial sequence
  Synthetic sequence Reverse primer
gtaccattatggcgtgtgaagtca

SEQ ID NO: 22 - DNA, 16 bp, Artificial sequence
  Synthetic sequence Probe
caccctggcacataaa

SEQ ID NO: 23 - DNA, 19 bp, Artificial sequence
  Synthetic sequence Forward primer
ccgtcttcccttccatcgt

SEQ ID NO: 24 - DNA, 20 bp, Artificial sequence
  Synthetic sequence Reverse primer
cccacgtaggagtccttctg

SEQ ID NO: 25 - DNA, 17 bp, Artificial sequence
  Synthetic sequence Probe
catcacgccctggtgcc

SEQ ID NO: 26 - DNA, 20 bp, Artificial sequence
  Synthetic sequence Forward primer
ccctagtcggggagagattt

SEQ ID NO: 27 - DNA, 20 bp, Artificial sequence
  Synthetic sequence Reverse primer
ttctatgccaccagagacca

SEQ ID NO: 28 - DNA, 505 bp, Ovis aries
  misc_feature (500)..(500): n is a, c, g, or t
ccctagtcggggagagatttttgacatttagagaaagaatgaatgcatatcgrgaagtggagtcagtagaaccccagaactgccaccttattgagggaattgaaaatggctctgaagatattgatatccttcctagtgggctggctttcatctccagtggattaaaatatccaggcatgccagactttgcaccagatragccaggacgaatcttcttgatggatttaaatgagcaaaaccctagggtccaggaattaaacatcagtgatggttttgacaaagcatcctttaatccacatggaatgagtacttycattgacaaagaccaaactgtgtatctttatgttgtgaatcatccccacatggrctccaccgtggagatatttaaatttgaggaacaacaacgttctttrgtatacctgaaaactataaaacatgaacttctcaaaagtgtgaatgatatcgtggttctcggaccagaagagttctatgccaccagngacca

SEQ ID NO: 29 - DNA, 505 bp, Bos taurus
ccctagtcggggagagatttttgacacttcgagaaagaatgaatgcatatcgagaagtggagtcagtagaatcccagaactgccaccttattgagggaattgaaaatggctctgaagatattgatatccttcctagtgggctggctttcatctccaatggattaaaatatccaggcatgccagaccttgcaccagatgagccaggacgaatcttcttgatggatttaaatgagcaaaaccctagggtccaggaattaaacatcagtgatggttttgacaaagcatcctttaatccacatggaatgagtactttcattgacaaagaccaaactgtatatctttatgttgtgaatcatccccacatggactccaccgtggagatatttaaatttgaagaacaacaacgttctttggtatacctgaaaactataaaacacgaacttctcaaaagtgtgaatgatattgtggttctcggaccagaacagttctatgccaccagagacca

SEQ ID NO: 30 - DNA, 505 bp, Homo sapiens
ccttagtcggggagatgttcctggcgtttagagaaagggtgaatgcctctcgagaagtggagccagtagaacctgaaaactgccaccttattgaggaacttgaaagtggctctgaagatattgatatacttcctagtgggctggcttttatctccagtggattaaaatatccaggcatgccaaactttgcgccagatgaaccaggaaaaatcttcttgatggatctgaatgaacaaaacccaagggcacaagcgctagaaatcagtggtggatttgacaaagaattatttaatccacatgggatcagtattttcatcgacaaagacaatactgtgtatctttatgttgtgaatcatccccacatgaagtccactgtggagatatttaaatttgaggaacaacaacgttctctggtatacctgaaaactataaaacatgaacttctcaaaagtgtgaatgacattgtggttcttggaccagaacagttctatgccaccagagacca

SEQ ID NO: 31 - protein, 354 aa, Bos taurus
MGKLMALILLGAGLALVGERFLTLRERMNAYREVESVESQNCHLIEGIENGSEDIDILPSGLAFISNGLKYPGMPDLAPDEPGRIFLMDLNEQNPRVQELNISDGFDKASFNPHGMSTFIDKDQTVYLYVVNHPHMDSTVEIFKFEEQQRSLVYLKTIKHELLKSVNDIVVLGPEQFYATRDHYFTNFFLVLLELFMDLRWTQVLFYSPKEVKVVARGFSSANGITISLDKKYIYVADVSDKNIHVMKIHDNWDLTQLKVIQLDTLVDNLTVDPDTGDILAGCHPNAIKLLMYNTEDPPGSEALRIRNALSEKPMISTMYANNGSVLQGSSVASVYRGKLLIGTVFHKALYCEL

SEQ ID NO: 32 - DNA, 2244 bp, Bos taurus
ccggagaccatggggaagctgatggcacttatcctgctgggcgccggcctggccctagtcggggagagatttttgacacttcgagaaagaatgaatgcatatcgagaagtggagtcagtagaatcccagaactgccaccttattgagggaattgaaaatggctctgaagatattgatatccttcctagtgggctggctttcatctccaatggattaaaatatccaggcatgccagaccttgcaccagatgagccaggacgaatcttcttgatggatttaaatgagcaaaaccctagggtccaggaattaaacatcagtgatggttttgacaaagcatcctttaatccacatggaatgagtactttcattgacaaagaccaaactgtatatctttatgttgtgaatcatccccacatggactccaccgtggagatatttaaatttgaagaacaacaacgttctttggtatacctgaaaactataaaacacgaacttctcaaaagtgtgaatgatattgtggttctcggaccagaacagttctatgccaccagagaccactattttaccaacttcttcttggtgctattagagttgttcatggatcttcgctggactcaggtgctcttctacagcccaaaagaggttaaagtggtggccagaggatttagttctgccaatggaattacaatctcactagacaagaagtatatctatgtagctgatgtatcagataagaacattcatgttatgaaaatacatgacaactgggatttaactcaactcaaggtaatccagttggacaccttagtggataacttaactgttgatcctgacacgggagatattttggcaggatgccatcctaatgctatcaagctgctgatgtataacaccgaggatcctccagggtcagaagcacttcgcatccggaacgctttgtctgagaagcccatgataagcaccatgtatgccaacaatggctcggtgcttcagggcagctctgtggcttctgtgtatagggggaagcttctcataggcactgtatttcacaaagccctgtactgtgagctctagactccagatattcccaaaagtctacacatttggtaaaagtaaacccctaatcatataagtagaactctaagtaaatgacaaacaccaataaaagtgtgtccagctttcctcatgcatagacataaagaatcagagacattacataatattcccctaattgcaagttaagttagtgacagtccaacaagaaaccaaacctctcaattctttaaccaactattcccaatattctttctacaataagacatgtgagctgtgatgagtgaggtgtccagcttacttggtagctgtgtggcaaaattacctcatacagctgtgctattctgagcctcctgcagtccagaccaggagaagagctaatgttcaaatatttacaattcaccttctcttccttcaattgcacgaggcacaaactgccagtcacgtgcccaacacatggataaaagcaagaactggcattgtgagactgcccaagccctccctgaactggcattctcccaatgcagacacacaggaagtcaaattctggaaaacctgacaaaagacagatggcactgtctactttgggccacagcagagctaatctgatctttgtaacaggtcattattcatggtggtatcttgtctctgaagtgaagtgttgttatcagcttgactctgaactaggagagagggcctcagaactgttaattctcagctgctttaaacttgttattttggttgattcagtaaacacttattaagcaacattccacagacttgaccctaaaaaatctgaaataaataacacagtatagtactgaagttctgtctaaggactcaaccttagtttgacgagaatgaatagaagtgtccctttcttggtcaaaaatcaagacaacctttagcaaatcattaacgtggcttagtatacccaagcaccccaccccctcccccttcccttttctgtgaattttactgagaagtggttggttgatatatttttctcttctgagggtggggtgttggggtgatttctttatgtcctaagtcattttgctcagttagatgtaagttttgttgaaaagttttgtagccaggtaaaattctacattttagcccatgtaaccctttctgatttagacaataaaactgtggtttccagaaaaaaaaaaaaaaaaaaaaaaaaaa

SEQ ID NO: 33 - protein, 354 aa, Sus scrofa
MGKLVALTLLGAFLALIGERLVALREKTNAYREVEPVEPQNCHLIEGLENGSEDIDILPSGLAFISTGLNYPGMVNFAPDKPGQIFLMDLNEPNPRVQALNISDGFDKASFNPHGISTFIDKDHTVYLYVVNHPHMESTVEIFKFEEQQRSLIHLKTIKHELLKSVNDIVVLGAEQFYATRDHYFTNFFLATLEIVLDLHWTYVLFYSPREVKVVAKGFNSANGITVSSDKKYIYVADVFDKNIHVMKKHDNWDLTPLKVIQLDTLVDNLTIDPDTGDIWAGCHPNAIKLVIYNTEDPPGSEVLRIQNALSEKPRISTEYANTGSVLQGSSVASVYKGKLLIGTVFHKALYCVL

SEQ ID NO: 34 - protein, 122 aa, Mus musculus
MKPSIAIILCILILGVDSQRWVQFMKEAGQGSRDMWRAYSDMKKANWKNSDKYFHARGNYDAARRGPGGAWAAKVISDAREAVQKFTGHGAEDSRADQFANEWGRSGKDPNHFRPAGLPKRY

SEQ ID NO: 35 - DNA, 531 bp, Mus musculus
tggggtcccagaaggagctcgcagcacgagcaggatgaagccttccattgccatcattctttgcatcttgatcctgggagttgacagccaaagatgggtccagttcatgaaagaagctggtcaagggtctagagacatgtggcgagcctactctgacatgaagaaagctaactggaaaaactcagacaaatacttccatgctcgggggaactatgatgctgcccggaggggtcccgggggagcctgggctgctaaagtcatcagcgatgccagagaggctgttcagaagttcacgggacatggagcagaggactcaagagctgaccagtttgccaatgagtggggccggagtggcaaagaccccaaccacttccgacctgctggcctgcctaaaagatactgagttttctcttcctgttgttcccagtcatgctgccccccgagaagaggagcaactactgggttgagatattttctaaaatctggatccctaaacatcccaatgtgctgaataaatacttgtgaaatgca

SEQ ID NO: 36 - DNA, 639 bp, Rattus norvegicus
gcatttcacaagcatttattcagcacattgggatgtttagggatccaggttttaggaaataactcaactcagtaattgcccctttccttggggggctgtgcgcccgggaacaacatgaagagaaaattcagtatttcctgggcagaccaacaggtcgaaagtggttggggtctttgccacttccggccccacttgttggcaaactggtcagctcttgagtcctctgctccgtgtcctgttaatctctgaataccctctctggcatcactgatgactttagcagcccaggctcccccaggaccccttcgggcggcatcatagttcccccgagcatggaagtatttgtctgagtttttccagttggcctttctcatgtccgagtaggcgcgccacatgtctttggtcccttgcccagcttccttcataaactggaaccgtcttttgctgtcaactcccaggatcaagaggcaaaaaacgatggcaatggaagacttcatcctgcttgtgctgtgaactcctgctgggaccccaggtgagtggcctctgtcccttgctgaggagatctggctggagacgtctatatgtactgccactgcccgtatggagcaatccccttttggtcttacgcaatcccccttg

SEQ ID NO: 37 - protein, 131 aa, Bos taurus
MNLSTGIIFCFLILGVSSQRWGTFLKEAGQGAKDMWRAYQDMKEANYRGADKYFHARGNYDAARRGPGGAWAAKVISNARETIQGITDPLFKGMTRDQVREDSKADQFANEWGRSGKDPNHFRPAGLPDKY

SEQ ID NO: 38 - DNA, 546 bp, Bos taurus
caccaggagcctcagcaggagggcacggccacaggatgaacctttccacgggcatcattttctgcttcctgatcctgggcgtcagcagccagagatgggggacattcctcaaggaagctggtcaaggggctaaagacatgtggagagcttaccaagacatgaaagaagccaactacaggggtgcagacaaatacttccacgcccgtggaaactatgacgctgcccgaaggggacctgggggtgcctgggctgctaaagtgatcagtaacgccagagagactattcagggaatcacagaccctctgtttaagggtatgaccagggaccaggtacgggaggattcgaaggccgaccagtttgccaacgaatggggccggagcggcaaagaccccaaccacttcagacctgctggcctgcctgacaagtactgagctgcctctctctctgctcaggagatgggctgtgagtccctaagggcagagacactgacctagagagttctctgtcctcagaaggcagcagatctaataaatgctcaagagatgg

(19) United States
(12) Patent Application Publication
(10) Pub. No.: US 2012/0207739 A1
(43) Pub. Date: Aug. 16, 2012

(54) TREATMENT OF PRE-TERM NEONATES

(75) Inventors: Gordon Smith, Cambridge (GB); David Stephen Charnock-Jones, Cambridge (GB)

(73) Assignee: Cambridge Enterprise Limited, Cambridge (GB)

(21) Appl. No.: 13/503,161

(22) PCT Filed: Oct. 22, 2010
(86) PCT No.: PCT/GB2010/001965

§ 371 Date: Apr. 20, 2012

Related U.S. Application Data
(60) Provisional application No. 61/253,936, filed on Oct. 22, 2009.
(60) Provisional application No. 61/323,929, filed on Apr. 14, 2010.

(51) Int. Cl. A61K 38/46; G01N 33/53; A61P 43/00

(52) U.S. Cl. 424/94.6; 435/7.1

(57) ABSTRACT

This invention relates to the treatment of pre-term neonates with paraoxonase 3 (PON3) or serum amyloid A like protein 3 (SAA3) to reduce or prevent the morbidity and mortality associated with prematurity.

This invention relates to the treatment of pre-term neonates, for example to reduce or prevent prematurity related morbidity and mortality.

Premature birth affects between 5-10% of the population in the developed world and around 25% of the population in the developing world.¹ Preterm birth accounts for the majority of neonatal deaths and for the vast majority of neonatal intensive care unit workload. This represents a significant burden of costs, with a single day of neonatal intensive care costing around £1000 and total annual costs in the UK estimated at £420 million in 2006-2007.²

One of the most successful interventions in neonatal intensive care is the administration of synthetic surfactant into the lungs of preterm infants immediately following birth. This reduces morbidity and mortality related to prematurity (see Soll³ for review). Surfactant was identified in animal studies where it was found to have a critical role in reducing surface tension in the lungs when the neonate takes its first breath. Expression of surfactant within the lung increases in the last third of pregnancy and in many species, including man, this is induced by corticosteroids in the fetus. The use of surfactants is an example of how understanding of the biology of preparation for birth may be translated into useful and profitable therapeutic interventions.

The present invention relates to the finding that transcripts encoding paraoxonase 3 (PON3) and serum amyloid A like protein 3 (SAA3) are up-regulated in the fetus in the final stages of normal mammalian gestation and play an important role in adapting the neonate to the changes that ensue after delivery from the maternal environment at the time of birth. Paraoxonase 3 (PON3) and/or serum amyloid A like protein 3 (SAA3) may therefore be useful as therapeutics in reducing or preventing morbidity or mortality in pre-term neonates

An aspect of the invention provides a method of treatment of a pre-term neonate comprising;
increasing the level or activity of paraoxonase 3 (PON3) polypeptide in the pre-term neonate.

Another aspect of the invention provides a method of treatment of a pre-term neonate comprising;
increasing the level or activity of serum amyloid A like protein 3 (SAA3) polypeptide in the pre-term neonate.

A pre-term neonate is an infant born before the end of the normal gestation period. In humans, infants delivered at a gestational age of less than 37 weeks (as calculated from the best clinical estimate of the estimated due date, where this is taken to equal 40 weeks and zero days) are generally considered to be pre-term.

In some embodiments, the pre-term neonate may be an extreme pre-term neonate, for example a human infant which is delivered at a gestational age of less than 32 weeks.

A pre-term neonate suitable for treatment as described herein may be a mammalian pre-term neonate, preferably a human pre-term neonate.

A pre-term neonate suitable for treatment as described herein may be deficient in PON3. In other words, the level or activity of PON3 in the pre-term neonate may be less than the level or activity of PON3 in full-term neonates. In some embodiments, the pre-term neonate may be identified as deficient in PON3 before treatment, either directly by measuring levels of PON3 before or after delivery, or indirectly by identifying one or more symptoms associated with PON3 deficiency, such as an oxidative stress related complication. In other embodiments, the pre-term neonate may be treated as described without being identified as deficient in PON3.

A pre-term neonate suitable for treatment as described herein may be deficient in SAA3. In other words, the level or activity of SAA3 in the pre-term neonate may be less than the level or activity of SAA3 in full-term neonates. In some embodiments, the pre-term neonate may be identified as deficient in SAA3 before treatment, either directly by measuring levels of SAA3 before or after delivery, or indirectly by identifying one or more symptoms associated with SAA3 deficiency, such as an oxidative stress related complication. In other embodiments, the pre-term neonate may be treated as described without being identified as deficient in SAA3.

A method of identifying a pre-term neonate at risk of morbidity and/or mortality may comprise:
determining the level or activity of PON3 and/or SAA3 in a sample of obtained from the neonate;
wherein a reduced level or activity of PON3 and/or SAA3 relative to controls is indicative that the pre-term neonate is at risk of morbidity and/or mortality.

Levels of PON3 and/or SAA3 may be determined in a sample obtained from the pre-term neonate, for example a blood or serum sample, by standard techniques, for example immunoassays such as ELISA or by assays of enzymatic activity.

A pre-term neonate at risk of morbidity and/or mortality may have an increased risk of morbidity and/or mortality relative to a full-term neonate. Pre-term neonates identified as being at risk of morbidity and/or mortality may be treated as described herein.

Increasing levels or activity of PON3 polypeptide and/or SAA3 polypeptide in the pre-term neonate as described herein may reduce the extent, severity or risk of morbidity and/or mortality. For example, treatment as described herein may prevent or reduce the severity or risk of medical complications of prematurity, for example, complications associated with oxidative stress.

Complications of prematurity which may be treated include respiratory complications, such as respiratory distress syndrome, pulmonary hypertension and bronchopulmonary dysplasia; neurological complications, such as apnea of prematurity, hypoxic-ischemic encephalopathy (HIE), intracranial hemorrhage, retinopathy of prematurity (ROP), developmental disability, and cerebral palsy; cardiovascular complications, such as patent ductus arteriosus (PDA); gastrointestinal and metabolic complications, such as hypoglycemia, feeding difficulties, and necrotizing enterocolitis (NEC); and hematologic complications, such as anemia of prematurity, thrombocytopenia, and hyperbilirubinemia (jaundice) and kernicterus.

In some embodiments, complications of prematurity which may be treated as described herein do not include sepsis or infection, or conditions associated with sepsis or infection.

The level or activity of paraoxonase 3 (PON3) polypeptide may be increased systemically in the neonate or locally in specific organs or tissues, for example in the lungs, vascular system and/or other tissues of the pre-term neonate.

Preferably, the level of the PON3 polypeptide is increased by administering PON3 polypeptide to the pre-term neonate.

Paraoxonase 3 (PON3; GeneID: 5446) is a phosphodiesterase (EC 3.1.8.1) which associates with high density lipoprotein in the serum.

The amino acid sequence of human PON3 has the Genbank database identifiers NP_{—}000931.1 GI: 29788996 and is shown in SEQ ID NO: 2. The nucleotide sequence encoding human PON3 has the Genbank database identifier NM_{—}000940.2 GI: 94538355 and is shown in SEQ ID NO: 1. The inventors have also identified a splice variant of human PON3 which has the amino acid sequence shown in SEQ ID NO: 4. The nucleotide sequence encoding the human PON3 splice variant is shown in SEQ ID NO: 3.

Other PON3 polypeptides suitable for use as described herein may comprise the amino acid sequence of PON3 from a non-human mammal, such as cow or pig, or may be a fragment, variant or allele thereof. The amino acid sequence of bovine PON3 (GeneID: 510953) has the Genbank database identifiers NP_{—}001068947.1 GI: 115496165. The nucleotide sequence encoding bovine PON3 has the Genbank database identifier NM_{—}001075479.1 GI: 115496164. The amino acid sequence of porcine PON3 (GeneID: 733674) has the Genbank database identifiers NP_{—}001038069.1 GI: 113205866.

A PON3 polypeptide suitable for use as described herein may comprise the amino acid sequence of a reference PON3 sequence, such as SEQ ID NO: 2 or SEQ ID NO: 4, or a fragment, allele or variant of the reference PON3 sequence. For example, an allele or variant of a reference PON3 sequence, such as SEQ ID NO: 2 or SEQ ID NO: 4, may comprise an amino acid sequence having at least 70%, at least 80%, at least 90%, at least 95% or at least 98% sequence identity to the a reference PON3 sequence.

A PON3 polypeptide may, for example, comprise an amino acid sequence which differs from a reference PON3 sequence, such as SEQ ID NO: 2 or SEQ ID NO: 4, by insertion, addition, substitution or deletion of 1 amino acid, 2, 3, 4, 5-10, 10-20 or 20-30 amino acids.

A PON3 polypeptide may be a fragment of a full-length PON3 amino acid sequence, such as SEQ ID NO: 2 or SEQ ID NO: 4 or a variant or allele of SEQ ID NO: 2 or SEQ ID NO: 4. A fragment is a truncated polypeptide consisting of fewer amino acids than the full-length sequence that retains paraoxonase 3 activity. Suitable fragments may comprise at least 100, at least 150, at least 200, at least 250 or at least 300 amino acids of the full-length sequence. Suitable fragments retain PON3 activity.

A PON3 polypeptide suitable for use as described herein may be encoded by the nucleotide sequence of SEQ ID NO: 1 or SEQ ID NO: 3 or an allele or variant of SEQ ID NO: 1 or SEQ ID NO: 3. For example, a PON3 polypeptide may be encoded by a nucleotide sequence having at least 70%, at least 80%, at least 90%, at least 95% or at least 98% sequence identity to the nucleotide sequence of SEQ ID NO: 1 or SEQ ID NO: 3.

A nucleotide sequence encoding a PON3 polypeptide may, for example, differ from SEQ ID NO: 1 or SEQ ID NO: 3 by insertion, addition, substitution or deletion of 1 nucleotide, 2, 3, 4, 5-10, 10-20 or 20-30 nucleotides.

Similarly, the level or activity of serum amyloid A like protein 3 (SAA3) polypeptide may be increased systemically in the pre-term neonate or locally in specific organs or tissues, for example in the lungs, vascular system and/or other tissues of the neonate.

Preferably, the level of the SAA3 polypeptide is increased by administering SAA3 polypeptide to the pre-term neonate.

The amino acid sequence of human SAA3 has the Genbank database identifiers AAO48437.1 GI: 28864698 and is shown in SEQ ID NO: 6. The nucleotide sequence encoding human SAA3 has the Genbank database identifiers AY209188.1 GI: 28864697 and is shown in SEQ ID NO: 5.

Other SAA3 polypeptides suitable for use as described herein may comprise the amino acid sequence of SAA3 from a non-human mammal, such as cow, rat or mouse, or may be a fragment, variant or allele thereof. The amino acid sequence of murine SAA3 (GeneID: 20210) has the Genbank database identifiers NP_{—}035445.1 GI:6755396. The nucleotide sequence encoding murine SAA3 has the Genbank database identifier NM_{—}011315.3 GI: 118130197. The nucleotide sequence encoding rat SAA3 has the Genbank database identifier BF282318 GI: 11213489 and hybridizes to Affymetrix probe 1392647_at on the Rat230_{—}2 array. The amino acid sequence of bovine SAA3 (GeneID: 281474) has the Genbank database identifiers NP_{—}851359.2 GI: 38566696. The nucleotide sequence encoding bovine SAA3 has the Genbank database identifier NM_{—}181016.3 GI: 38566695.

A SAA3 polypeptide suitable for use as described herein may comprise the amino acid sequence of a reference SAA3 sequence, such as SEQ ID NO: 6, or a fragment, allele or variant of the reference SAA3 sequence. For example, an allele or variant of a reference SAA3 sequence, such as SEQ ID NO: 6, may comprise an amino acid sequence having at least 70%, at least 80%, at least 90%, at least 95% or at least 98% sequence identity to the a reference SAA3 sequence.

A SAA3 polypeptide may, for example, comprise an amino acid sequence which differs from a reference SAA3 sequence, such as SEQ ID NO: 6, by insertion, addition, substitution or deletion of 1 amino acid, 2, 3, 4, 5-10, 10-20 or 20-30 amino acids.

A SAA3 polypeptide may be a fragment of a full-length SAA3 amino acid sequence, such as SEQ ID NO: 6 or a variant or allele of SEQ ID NO: 6. A fragment is a truncated polypeptide consisting of fewer amino acids than the full-length sequence that retains serum amyloid A like protein 3 activity. Suitable fragments may comprise at least 30, at least 40, or at least 50 amino acids of the full-length sequence. Suitable fragments retain serum amyloid A like protein 3 activity.

A SAA3 polypeptide suitable for use as described herein may be encoded by the nucleotide sequence of SEQ ID NO: 5 or an allele or variant of SEQ ID NO: 5. For example, a SAA3 polypeptide may be encoded by a nucleotide sequence having at least 70%, at least 80%, at least 90%, at least 95% or at least 98% sequence identity to the nucleotide sequence of SEQ ID NO: 5.

A nucleotide sequence encoding a SAA3 polypeptide may, for example, differ from SEQ ID NO: 5 by insertion, addition, substitution or deletion of 1 nucleotide, 2, 3, 4, 5-10, 10-20 or 20-30 nucleotides.

Nucleotide and amino acid sequence identity is generally defined with reference to the algorithm GAP (GCG Wisconsin Package™, Accelrys, San Diego Calif.). GAP uses the Needleman & Wunsch algorithm³⁶—to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST or TBLASTN (which use the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), generally employing default parameters.

One or more heterologous amino acids, for example a heterologous peptide or heterologous polypeptide sequence, may be joined or fused to a PON3 or SAA3 amino acid sequence described herein. For example a PON3 polypeptide may comprise a PON3 amino acid sequence as described above linked or fused to one or more heterologous amino acids. The one or more heterologous amino acids may include sequences from a source other than PON3. Similarly, an SAA3 polypeptide may comprise an SAA3 amino acid sequence as described above linked or fused to one or more heterologous amino acids. The one or more heterologous amino acids may include sequences from a source other than SAA3.

A PON3 or SAA3 polypeptide may be comprised within a fusion protein.

In some embodiments, the fusion protein may be processed to produce the mature PON3 or SAA3 polypeptide before use in the treatment of pre-term infants as described herein. For example, in addition to the PON3 or SAA3 polypeptide, a fusion protein may comprise one or more heterologous amino acids at the N or C terminal which form all or part of a site-specific protease recognition sequence. The mature PON3 or SAA3 polypeptide may be produced from the fusion protein by cleavage of the site-specific protease recognition sequence, for example using a site-specific protease, such as thrombin or factor Xa.

The fusion protein may comprise a purification tag which is removed by the site-specific protease after purification. Suitable purification tags include glutathione-S-transferase (from Schistosoma japonica).

PON3 and SAA3 polypeptides may be produced by any convenient technique and a range of suitable approaches are available.

PON3 and SAA3 polypeptides may be isolated and/or purified from non-human mammals, such as pigs or cows.

PON3 and SAA3 polypeptides may be generated wholly or partly by chemical synthesis. For example, polypeptides may be synthesised using liquid or solid-phase synthesis methods; in solution; or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Chemical synthesis of polypeptides is well-known in the art (J. M. Stewart and J. D. Young, Solid Phase Peptide Synthesis, 2nd edition, Pierce Chemical Company, Rockford, Ill. (1984); M. Bodanzsky and A. Bodanzsky, The Practice of Peptide Synthesis, Springer Verlag, New York (1984); J. H. Jones, The Chemical Synthesis of Peptides. Oxford University Press, Oxford 1991; in Applied Biosystems 430A Users Manual, ABI Inc., Foster City, Calif.; G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997).

PON3 and SAA3 polypeptides may be generated wholly or partly by recombinant techniques. For example, a nucleic acid encoding a PON3 or SAA3 polypeptide may be expressed in a host cell and the expressed polypeptide isolated and/or purified from the cell culture.

For the production of recombinant PON3 or SAA3 polypeptide, nucleic acid sequences encoding the PON3 or SAA3 polypeptide (i.e. PON3 nucleic acid or SAA3 nucleic acid) may be comprised within an expression vector. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the PON3 or SAA3 nucleic acid in a host cell. Suitable regulatory sequences to drive the expression of heterologous nucleic acid coding sequences in a range of expression systems are well-known in the art and include constitutive promoters, for example viral promoters such as CMV or SV40, and inducible promoters, such as Tet-on controlled promoters. A vector may also comprise sequences, such as origins of replication and selectable markers, which allow for its selection and replication and expression in bacterial hosts such as E. coli and/or in eukaryotic cells, such as yeast, insect or mammalian cells.

Vectors suitable for use in expressing PON3 or SAA3 nucleic acid include plasmids and viral vectors e.g. ‘phage, or phagemid, and the precise choice of vector will depend on the particular expression system which is employed. PON3 or SAA3 polypeptide may be expressed in any convenient expression system, and numerous suitable systems are available in the art, including bacterial, yeast, insect or mammalian cell expression systems. For further details see, for example, Molecular Cloning: a Laboratory Manual: 3rd edition, Russell et al., 2001, Cold Spring Harbor Laboratory Press. Techniques and protocols for expression of recombinant polypeptides in cell culture and their subsequent isolation and purification are well known in the art (see for example Protocols in Molecular Biology, Second Edition, Ausubel et al. eds. John Wiley & Sons, 1992; Recombinant Gene Expression Protocols Ed R S Tuan (March 1997) Humana Press Inc).

As described above, the PON3 or SAA3 polypeptide may be expressed in an expression system as a fusion protein comprising the PON3 or SAA3 polypeptide sequence and a purification tag. Preferably, a protease recognition site is located between the PON3 or SAA3 polypeptide and the purification tag. Following expression, the fusion protein may be isolated by affinity chromatography using an immobilised agent which binds to the purification tag. After isolation, the fusion protein may be proteolytically cleaved, for example using thrombin or factor Xa, to produce the PON3 or SAA3 polypeptide.

A purification tag is a heterologous amino acid sequence which forms one member of a specific binding pair. Polypeptides containing the purification tag may be detected, isolated and/or purified through the binding of the other member of the specific binding pair to the polypeptide. For example, the purification tag may form an epitope which is bound by an antibody molecule.

Various suitable purification tags are known in the art, including, for example, MRGS(H)₆, DYKDDDDK (FLAG™), T7-, S- (KETAAAKFERQHMDS), poly-Arg (R₅₋₆), poly-His (H₂₋₁₀), poly-Cys (C₄) poly-Phe (F₁₁) poly-Asp(D₅₋₁₆), Strept-tag II (WSHPQFEK), c-myc (EQKLISEEDL), Influenza-HA tag (Murray, P. J. et al (1995) Anal Biochem 229, 170-9), Glu-Glu-Phe tag (Stammers, D, K. et al (1991) FEBS Lett 283, 298-302), Tag.100 (Qiagen; 12 aa tag derived from mammalian MAP kinase 2), Cruz tag 09™ (MKAEFRRQESDR, Santa Cruz Biotechnology Inc.) and Cruz tag 22™ (MRDALDRLDRLA, Santa Cruz Biotechnology Inc.). Known tag sequences are reviewed in Terpe (2003) Appl. Microbiol. Biotechnol. 60 523-533.

In some preferred embodiments, a glutathione-S-transferase purification tag may be employed. Following expression, a fusion protein comprising the PON3 or SAA3 polypeptide and glutathione-S-transferase may be isolated by affinity chromatography using immobilised glutathione. The purification of glutathione-S-transferase fusion proteins is well known in the art. After isolation, the fusion protein may then be proteolytically cleaved to produce the PON3 or SAA3 polypeptide.

PON3 and/or SAA3 polypeptides may be administered to pre-term neonates as described above. While it is possible for the PON3 or SAA3 polypeptide to be administered alone, it is preferable to present it as a pharmaceutical composition (e.g. a formulation) comprising the PON3 polypeptide and/or SAA3 polypeptide as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Pharmaceutical compositions comprising a PON3 polypeptide and/or a SAA3 polypeptide admixed or formulated together with one or more pharmaceutically acceptable carriers, excipients, buffers, adjuvants, stabilisers, or other materials, as described herein, may be used in the methods described above.

Another aspect of the invention provides a method of preparing a pharmaceutical composition comprising
providing an PON3 polypeptide and/or an SAA3 polypeptide as described above and
admixing the PON3 polypeptide and/or the SAA3 polypeptide with a pharmaceutically acceptable excipient.

The term “pharmaceutically acceptable” as used herein pertains to compounds, materials, compositions, and/or dosage forms which are, within the scope of sound medical judgement, suitable for use in contact with the tissues of a subject (e.g., human or other mammal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well-known in the art of pharmacy. Such methods include the step of bringing the PON3 polypeptide and/or SAA3 polypeptide into association with a carrier which may constitute one or more accessory ingredients. In general, the formulations are prepared by uniformly and intimately bringing into association the active compound with liquid carriers or finely divided solid carriers or both, and then if necessary shaping the product. Preferred formulations may be in the form of liquids or solutions, or slow release implants or capsules, for example for oral administration.

The PON3 polypeptide, SAA3 polypeptide, or pharmaceutical composition comprising the PON3 polypeptide and/or SAA3 polypeptide may be administered to a subject by any convenient route of administration. In some embodiments, administration is by parenteral routes, such as intravenous, sub-cutaneous, intrathecal or intratracheal routes. For example, the PON3 polypeptide and/or SAA3 polypeptide may be administered by injection, in particular intravenous injection. In other embodiments, administration is by enteral routes, for example, through a nasal jejunal tube or endotracheal tube.

Formulations suitable for parenteral administration (e.g. by injection, including intravenous), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Typically, the concentration of the active compound in the solution is from about 1 μg/ml to about 100 mg/ml, for example, from about 10 μg/ml to about 50 mg/ml.

Formulations suitable for enteral administration (e.g. by ingestion) may be presented as discrete units such as capsules, cachets or tablets, each containing a predetermined amount of PON3 polypeptide and/or SAA3 polypeptide; as a powder or granules; as a solution or suspension in an aqueous or non-aqueous liquid; or as an oil-in-water liquid emulsion or a water-in-oil liquid emulsion; as a bolus; as an electuary; or as a paste. Tablets or capsules may optionally be provided with an enteric coating, to provide release in parts of the gut other than the stomach.

Formulations comprising PON3 polypeptide and/or SAA3 polypeptide may be presented in unit-dose or multi-dose sealed containers, for example, ampoules and vials, and may be stored in a freeze-dried (lyophilised) condition requiring only the addition of the sterile liquid carrier, for example water for injections, immediately prior to use.

It will be appreciated that appropriate dosages of the PON3 polypeptide and/or SAA3 polypeptide, and pharmaceutical compositions comprising the PON3 polypeptide and/or SAA3 polypeptide, can vary from patient to patient, depending on the circumstances. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the PON3 polypeptide and/or SAA3 polypeptide, other drugs, compounds, and/or materials used in combination, and the maturity, sex, weight, condition and general health of the pre-term infant. The amount of PON3 polypeptide and/or SAA3 polypeptide and route of administration will ultimately be at the discretion of the physician, although generally the dosage will be to achieve serum concentrations of the PON3 polypeptide and/or SAA3 polypeptide which are sufficient to produce a beneficial effect without causing substantial harmful or deleterious side-effects.

Administration in vivo can be effected in one dose, continuously or intermittently (e.g., in divided doses at appropriate intervals). Methods of determining the most effective means and dosage of administration are well known to those of skill in the art and will vary with the formulation and the subject being treated. Single or multiple administrations may be carried out with the dose level and pattern being selected by the physician.

PON3 polypeptide and/or SAA3 polypeptide are preferably administered immediately or shortly after delivery of the pre-term neonate. For example, the PON3 polypeptide and/or SAA3 polypeptide may be administered less than 1 hour, less than 3 hours, less than 6 hours, less than 12 hours, less than 24 hours or less than 48 hours after delivery.

In other embodiments, the PON3 polypeptide and/or SAA3 polypeptide may be administered more than 48 hours after birth.

The PON3 polypeptide may be administered prophylactically after delivery or when the pre-term neonate displays one or more symptoms associated with PON3 deficiency, such as oxidative-stress related complications.

The SAA3 polypeptide may be administered prophylactically after delivery or when the pre-term neonate displays one or more symptoms associated with SAA3 deficiency.

In some embodiments, a SAA3 polypeptide may be administered to a pre-term neonate who is not subject to mechanical ventilation and/or lung injury or inflammation.

The PON3 polypeptide and/or SAA3 polypeptide, and compositions comprising the PON3 polypeptide and/or SAA3 polypeptide may be administered in combination with other therapies, such as surfactants and steroids, as described above.

Other aspects of the invention provide a PON3 polypeptide and/or a SAA3 polypeptide as described above for use in the treatment of a pre-term neonate, and the use of a PON3 polypeptide and/or a SAA3 polypeptide in the manufacture of a medicament for use in the treatment of a pre-term neonate. Treatment of pre-term neonates using the PON3 polypeptide and/or the SAA3 polypeptide may reduce the extent, severity or risk of morbidity and/or mortality, as described above.

Various further aspects and embodiments of the present invention will be apparent to those skilled in the art in view of the present disclosure. All documents mentioned in this specification are incorporated herein by reference in their entirety.

The sequences having the database (e.g. Genbank) accession numbers set out above at the filing date are also incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described below.

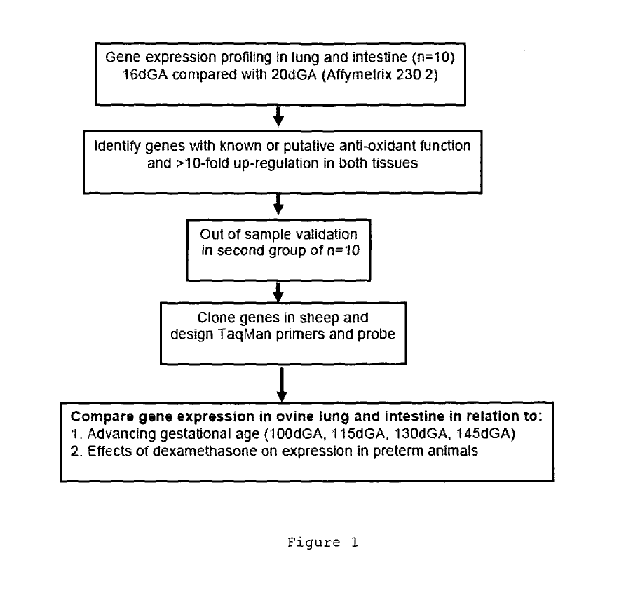

FIG. 1 shows the experimental scheme for identifying transcripts up or down-regulated in the foetus in late pregnancy.

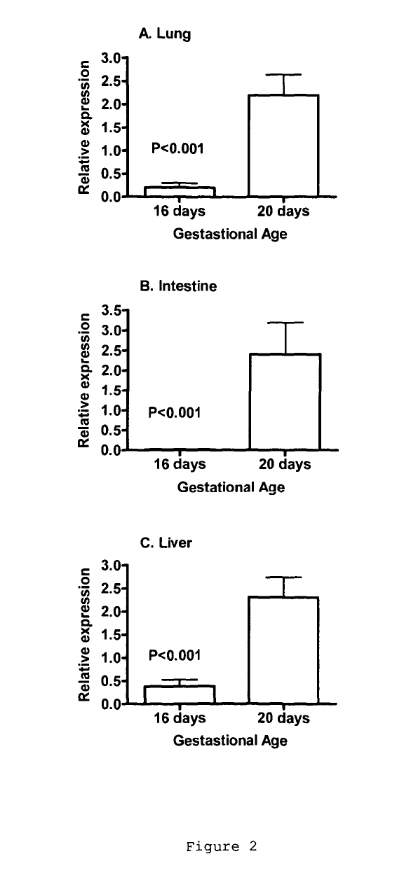

FIG. 2 shows the results of RT-PCR analysis of Pon3 mRNA relative to 18S RNA in lung tissue (A), intestine (B) and liver (C) in siblings of rats used in initial array, comparing rats delivered at 16 and 20 days of gestational age (both n=10, each animal from a separate litter). P value compares log transformed Pon3 transcript levels (relative to 18S RNA) using Student's unpaired t test at the two gestational ages in a given tissue.

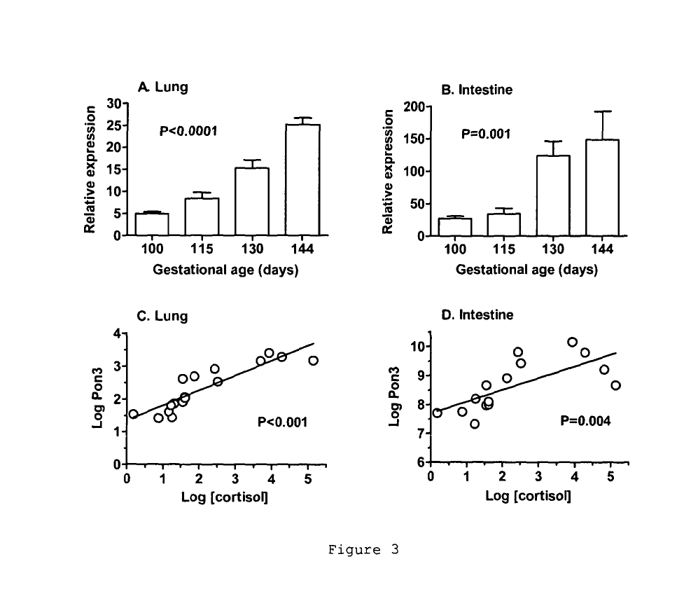

FIG. 3 shows the results of the RT-PCR analysis of PON3 mRNA relative to the geometric mean of four housekeeping genes, in sheep lung (A) and intestine (B) at different gestational ages in the last third of pregnancy (n=5 in each group). Columns=means; bars=SEM. P value from linear regression of log transformed Pon3 transcript levels (relative to four housekeeping genes) versus gestational age. Spearman's rho 0.94 for A and 0.79 for B, both P<0.001. Panels C and D are log transformed Pon3 levels plotted against log transformed cortisol values. P values from linear regression of log transformed Pon3 levels versus log transformed cortisol. Spearman's rho 0.94 for C and 0.80 for D, both P<0.001.

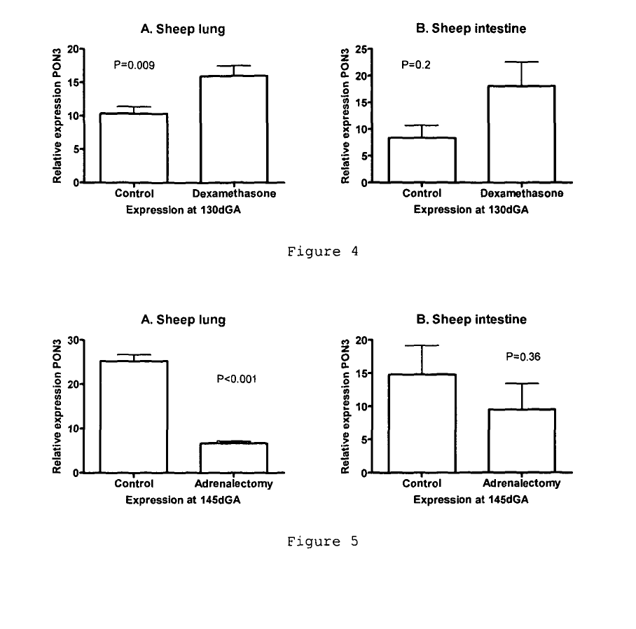

FIG. 4 shows the results of RT-PCR analysis of PON3 mRNA in the lung and intestine of dexamethasone treated and control sheep at 130 days gestation (n=5 in each group). Columns=means; bars=SEM. Levels of PON3 mRNA are shown relative to the geometric mean of four housekeeping genes.

FIG. 5 shows the results of RT-PCR analysis of PON3 mRNA in the lung and intestine of sheep which had preterm (115-188 dGA) adrenalectomy and control sheep, both groups delivered at 145 days gestation (n=5 in each group). Columns=means; bars=SEM. Levels of PON3 mRNA are shown relative to the geometric mean of four housekeeping genes.

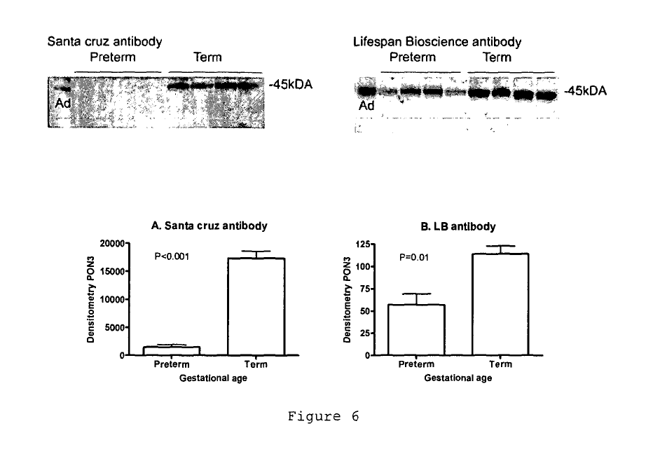

FIG. 6 shows western blot analysis of PON3 in adult blood (Ad), and umbilical cord blood from 4 preterm and 4 term infants, comparing using different commercially available antibodies (top panel). Also shown are the results of densitometry on signals comparing term and preterm (lower panel).

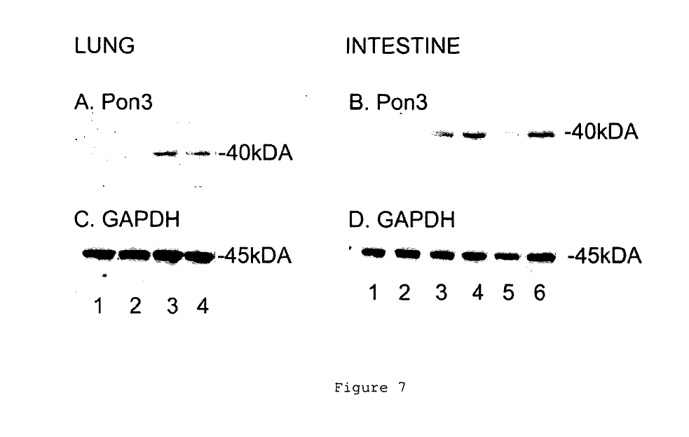

FIG. 7 shows western blot analysis of rat lung (A and C) intestine (B and D) at 16 and 20 days of gestational age. Panels A and B employed an antibody specific for PON3. Panel C and D employed the same blots as A and B, respectively, stripped and re-probed with an antibody specific for GAPDH. Panels A and C: lanes 1 and 2 lungs from 16 dGA and lanes 3 and 4 lungs from 20 dGA. Panels B and D: lanes 1 and 2=combined small and large intestines from 16 dGA; lanes 3 and 4=small intestines from 20 dGA; Lanes 5 and 6=large intestines from 20 dGA.

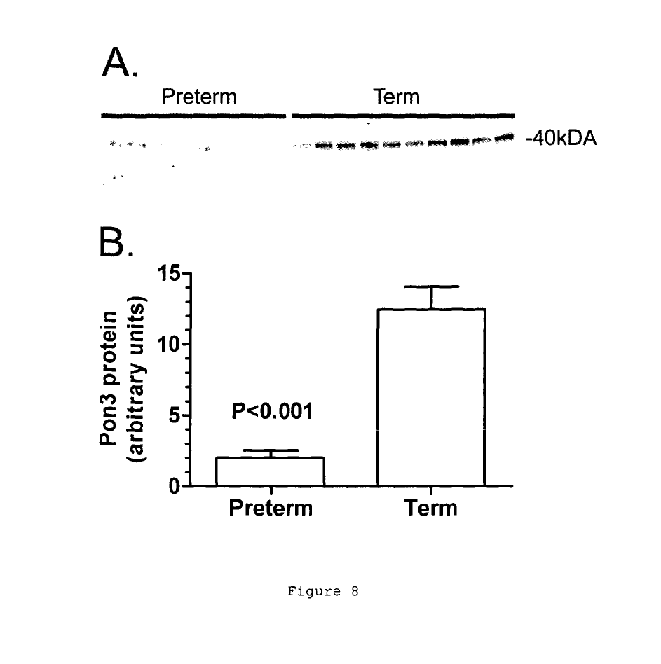

FIG. 8A shows western blot analysis of PON3 in umbilical cord serum from 8 preterm and 10 term infants. FIG. 8B shows results of densitometry on signals comparing term and preterm. P value of log transformed densitometry values comparing preterm and term using Student's unpaired t test.

Table 1 shows the primers and probes used for real-time RT-PCR in sheep

Table 2 shows the fold changes from gene array between 16 and 20 days of gestational age (dGA) for selected transcripts.

### EXPERIMENTS

### Methods

### Animal Samples

All experimental procedures were carried out in accordance with the UK Animals (Scientific Procedures) Act 1986 under an appropriate Home Office License and with approval from the local ethical review process.

Twenty time-mated pregnant Wistar rats were euthanized by CO₂ overdose at 16 (n=10) and 20 days of gestational age (dGA) n=10), where day 0 was defined as the day of plug and term is 21 days. Pups were delivered by caesarean section, euthanized by cervical dislocation, and their organs were removed under a dissecting microscope. Tissues from multiple siblings were snap frozen in liquid nitrogen prior to storage at −80° C. for subsequent analysis. Experiments in sheep were conducted as previously described in detail.⁷ In brief, pregnant Welsh Mountain ewes of known gestational age were kept in individual pens and maintained on 200 g/d concentrates with free access to hay, water, and a saltlick block. To study the effect of gestational age on gene expression, normal fetuses were delivered for tissue collection by caesarean section under general anesthesia (20 mg/kg sodium pentobarbitone i.v.) at 100 (n=5), 114-116 (n=5), 129-131 (n=5) and 144-145 (n=5) days of gestation where term is 145±2 days. To study the effect of preventing the normal rise in endogenous corticosteroids that occurs at term, fetuses (n=5) were adrenalectomized under general anesthesia (1.5% halothane in 1:1 O₂:N₂O) between days 116-120 dGA and then delivered at term. This has previously been shown to reduce cortisol levels to 15% of normal term levels. To study the effect of maternal glucocorticoid treatment, ewes were treated with either two doses of 12 mg dexamethasone or saline 24 hours apart starting 125 dGA (n=5 each) and fetuses were delivered 10 hours after the second injection by caesarean section. In all cases, newborn lambs were euthanized with sodium pentobarbitone (200 mg/kg) immediately after delivery. Umbilical cord blood was obtained following delivery. Cortisol concentrations were measured by radioimmunoassay validated for use with ovine plasma.²⁰ The minimum detectable quantity of cortisol was 1.5 ng/ml and interassay coefficient of variation was 11%. During necropsy, samples of fetal lung and jejunum (midway between the pyloric sphincter and ileo-caecal junction) were frozen in liquid nitrogen and stored at −80° C. until analysis.

### Human Cord Blood Samples

Protocols for obtaining human cord blood were approved by Cambridge Local Research Ethics Committee 2. All women provided written informed consent. Pre-term infants were eligible for study if they were liveborn between 24 and 28 completed weeks of gestational age. Term was defined as ≧37 weeks.

RNA Extraction and cDNA Synthesis

Total RNA was isolated from frozen tissues using TRIzol® (Invitrogen, Paisley, UK) according the manufacturer's instructions and treated with DNaseI (Promega, Southampton, UK) for 30 minutes at 37° C. The reaction was stopped by addition of Stop solution and heating at 65° C. for 10 minutes. RNA integrity was confirmed using a spectrophotometer and an Agilent 2100 Bioanalyzer. Only RNA with an OD 260/280>1.8 was used for quantitative PCR and RNA with a RNA integrity number greater than 7 was used for array hybridization. For quantitative RT-PCR, total RNA was reverse transcribed to cDNA using random hexamer primers (Bioline, London, UK) and Superscript II Reverse Transcriptase (Invitrogen, Paisley, UK).

### Microarray Analysis

Affymetrix Rat Genome 230.2 GeneChip was employed. Total RNA was processed using the standard Affymetrix two-cycle target labeling and hybridization protocols. Data were pre-normalized using robust multiarray averaging and normalization was achieved using the LIMMA software package (linear models of microarray data, http://bioinf.wehi.edu.au/limma/). Normalized transcript abundance data were compared between 16 dGA and 20 dGA using two independent methods: the Cyber-T algorithm and Rank Product Analysis.^{21, 22} The Cyber-T algorithm is an unpaired t-test, modified by the inclusion of a Bayesian prior based on the variance of other transcripts in the data set. Transcripts that were statistically significant using both Cyber-T (Bayes P value <0.001, posterior probability of differential expression>0.99) and Rank Product Analysis (P<0.0001) were defined as differentially expressed. Microarray data were annotated using the NetAffx™ Analysis Center (Affymetrix).

Quantitative Real-Time RT-PCR of Rat cDNA

All real-time PCR was done on an ABI Prism 7900HT system (Applied Biosystems, Warrington, UK) and performed in 10 μl volume containing 100 ng cDNA as template, 1×ABI PCR Mix No AmpErase UNG (Applied Biosystems, Warrington, UK) and pre-designed and pre-optimized gene expression assays for paraoxonase 3 (Pon3) and 18S purchased from Applied Biosystems, Rn01500926_ml and Rn00824548_ml, respectively. Sequences of these proprietary primer-probe sets are not available. PCR cycles included an initial denaturation at 95° C. for 20 seconds followed by 40 cycles of 95° C. for 1 seconds and 60° C. for 20 sec. All samples were analyzed in triplicate. Expression levels were quantified relative to 18S using the ddCt method.

Sheep cDNA Cloning and Quantitative Real-Time RT-PCR

As the sequence of the ovine Pon3 gene has not yet been published, we searched the NCBI expressed sequence tag (EST) database using the bovine Pon3 mRNA (nm_{—}001075479). This identified several sheep ESTs which showed >95% identity to the bovine gene. We used two ESTs showing the highest homology (EE859920 and EE792195) to design a PCR strategy in order to clone a fragment of the putative sheep Pon3 cDNA. The primers were as follows: forward (5′-CCCTAGTCGGGGAGAGATTT-3′) and reverse (5′-TTCTATGCCACCAGAGACCA-3′). The template used was cDNA generated from fetal sheep intestine. PCR was performed in 50 μl containing a final concentration of 1.25 mM MgCl₂, 200 μM of each dNTPs, 800 nM of each primers and 0.1 units/μl BioTaq™ DNA polymerase (Bioline, London, UK). The PCR program consisted of an initial denaturation at 95° C. for 5 minutes followed by 40 cycles of 95° C. for 45 sec, 60° C. for 45 sec, 72° C. for 60 seconds and a final extension step of 72° C. for 10 minutes. PCR products were analyzed on a 1.5% agarose gel and bands of the expected size were excised and purified using the Qiaquick™ Gel Extraction Kit (Qiagen, Crawley, UK) then cloned into the pGEM-T-Easy™ plasmid (Promega). Three clones were sequence verified.

Real-time PCR assays with 6-FAM labeled major groove binder (MGB) probe were designed with Applied Biosystems File Builder 3.1 using the cloned partial cDNA for ovine PON3 and publicly available sequence data (NCBI) for ovine housekeeping genes (Table 1). Relative quantification of Pon3 was performed against the geometric mean of four housekeeping genes (18S, GAPDH, cyclophilin A and β-actin) as we observed greater inter-animal variability in the sheep and this method has been reported to be superior to using any single reference gene.²³ PCR was performed in 10 μl containing 100 ng cDNA as template, 1× TaqMan Fast Universal PCR Master Mix No AmpErase UNG (Applied Biosystems, Warrington, UK) and the custom designed primer-probe sets. PCR cycles included an initial denaturation at 95° C. for 20 seconds followed by 40 cycles of 95° C. for 1 seconds and 60° C. for 20 seconds. A relative standard curve was generated for each assay using serial dilution of a reference sample and results were analyzed using the relative standard curve method.

### Western Blotting

To analyze Pon3 protein expression in rat intestines, frozen tissue was homogenized in 1×RIPA buffer (Millipore) containing 1 Complete Mini EDTA free Protease Inhibitor Cocktail Tablet per 10 ml (Roche) using a glass homogenizer. Protein concentration was determined using the BioRad DC protein assay. Samples containing 20 μg protein were reduced by adding Nupage Sample Reducing agent (Invitrogen), heat-denatured and loaded on a 10% Novex Bis-Tris Gel (Invitrogen). After electrophoresis, proteins were transferred to a PVDF membrane (Invitrogen), blocked with 5% skimmed milk in TBS-tween-20 (0.05%) for 1 hour and subsequently probed with a goat polyclonal anti-human Pon3 antibody (Lifespan Biosciences) at 0.2 μg/ml on a shaker at 4° C. overnight. Antibody binding was detected by horseradish peroxidase-conjugated rabbit anti-goat secondary antibody (DAKO). ECL substrate (Amersham Biosciences) was then used to visualize binding. Membranes were stripped with Restore™ Western stripping buffer (Pierce) for 15 minutes at room temperature and re-probed with a rabbit polyclonal antibody to human GAPDH (Abcam) at 0.2 μg/ml to confirm equal loading of protein.

To analyze Pon3 expression in the human fetus, cord blood samples were collected in lithium-heparin tubes and separated by centrifugation for 10 minutes at 3000 rpm at 4° C. The serum samples were diluted 1 in 10 in PBS, reduced by adding Nupage Sample Reducing agent (Invitrogen) and heat-denatured. 3 μl of the 1:10 diluted samples were loaded on a 10% Novex Bis-Tris Gel (Invitrogen). After electrophoresis, proteins were transferred to a PVDF membrane (Invitrogen), blocked with 5% skimmed milk in PBS-tween20 (0.05%) for 1 hour and subsequently probed with an antibody to human Pon3 (Lifespan Biosciences, LB), as described above. Re-probing of the Western to confirm equal loading was unnecessary as all lanes were loaded with identical volumes of serum. Hence, there is no noise related to variability of efficiency of extraction of protein or measurement of protein concentrations. Total protein was assessed by staining the blot with Ponceau s.

### Statistics

Transcript and cortisol levels were log transformed and means were then compared using Student's unpaired t-test. The association between two continuous variables was assessed using both Spearman rank correlation and linear regression. Statistical significance was assumed at P<0.05 and all P values were two sided.

### Summary of Results

We studied lungs and intestines in rat and sheep, which are phylogenetically only distantly related. Up-regulation in both tissues in both mammals is indicative that the gene is important in systemic preparation for birth in mammals generally, including humans, for example in defence against oxidative stress.

We studied a range of gestational ages and also studied the effects of corticosteroids in the sheep, as these are involved in up-regulating many of the key processes in the fetus which are preparative for birth.

The experimental scheme is shown in FIG. 1.

The array experiments demonstrated up-regulation between day 16 and 20 of the mRNA encoding paraoxonase 3 in both the lung (24-fold) and intestine (31-fold). Out of sample validation using quantitative TaqMan RT-PCR confirmed changes of comparable magnitude in lung and intestine from a separate series of animals (FIGS. 2A and 2B) and also in liver (FIG. 2C).

We then cloned a fragment of the PON3 cDNA in the sheep, designed primers and a probe for RT-PCR and analysed level of PON3 mRNA in 4 groups each with 5 animals delivered in the last third of gestation, using quantitative TaqMan RT-PCR. Samples of small intestine were obtained from the midpoint between the pyloric sphincter and the ileo-caecal junction. PON3 mRNA increased linearly with advancing gestational age and the changes in both tissues were highly statistically significant. An approximate 5-fold up-regulation was observed in both lung and intestine (FIGS. 3A and 3B).

We studied expression of PON3 in preterm sheep fetuses exposed to exogenous synthetic corticosteroid (dexamethasone) and vehicle. This demonstrated that corticosteroids increased PON3 mRNA in both the lung and the intestine, although, due to greater variability between samples, the difference was not statistically significant in the intestine (FIGS. 4A and 4B).

We then examined the effect of endogenous corticosteroids on PON3 mRNA level among animals delivered at term (145 days). Control animals were compared with those where the late gestation increase in endogenous corticosteroids was prevented by adrenalectomy, which was performed around days 115-118 of gestational age. Adrenalectomy was associated with lower PON3 mRNA in both tissues at term, although the difference was again not statistically significant in the intestine due to greater variability between samples (FIGS. 5A and 5B).

These results show that PON3 mRNA is up-regulated prior to birth in both the lung and intestine in both rats and sheep.

We then compared the levels of PON3 circulating in the preterm and term human fetus by obtaining umbilical cord blood obtained at the time of birth in 4 preterm infants and 4 infants born at term. PON3 protein levels in term babies were comparable with the adult, but levels of PON3 expression were lower in the cord blood of infants born preterm (FIG. 6).

### Pon3 Expression in Fetal Rats in Late Gestation

Microarray analysis demonstrated four genes which were up-regulated greater than 20-fold on the array between 16 and 20 dGA in both the lung and intestine (Table 2): serum amyloid A like protein 3, paraoxonase-3 (Pon3), solute carrier family 34, member 2, and chloride intracellular channel 5. In contrast, the expression of the other two members of the Pon gene family (Pon1 and Pon2) did not change significantly.

We analyzed expression of Pon3 by real-time quantitative RT-PCR in siblings of the animals used in the array and confirmed up-regulation of Pon3 mRNA between 16 dGA and 20 dGA in the rat lungs, intestines and liver (FIG. 2). Western analysis of 16 dGA and 20 dGA rat lung and intestine demonstrated that Pon3 protein was undetectable at 16 dGA, but there was a clear band of the appropriate molecular weight at 20 dGA (FIG. 7).

### Pon3 Expression in Fetal Sheep

As there was no published sequence for the ovine PON3 gene, we cloned a partial cDNA of the orthologous ovine gene. We obtained a 505 bp sequence that shared 96% homology with bovine Pon3 and 87% homology with human Pon3 at the nucleotide level. In contrast, the sequence showed less homology with bovine PON1 (62%) and PON2 (70%). A quantitative RT-PCR strategy was designed using the cloned sequence (Table 2). We then analyzed expression of PON3 in 4 groups each with 5 animals delivered in the last third of gestation. PON3 expression increased linearly with advancing gestational age (FIGS. 3A and 3B) and cortisol levels (FIGS. 3C and 3D) in both the lungs and intestines and all changes were highly statistically significant.

We then compared preterm sheep fetuses exposed to exogenous synthetic glucocorticoid (dexamethasone) or vehicle. This demonstrated that dexamethasone increased expression of PON3 in both the lung and the intestine, although, due to greater variability between samples, the difference was not statistically significant in the intestine (FIGS. 4A and B). We then examined the effect of blocking the normal late gestation rise of endogenous glucocorticoids on the expression of PON3 among animals delivered at term (145 days) by comparing control term animals with term animals where fetal adrenalectomy had been performed at 115-118 dGA. Adrenalectomy was associated with lower expression of PON3 in both tissues at term, although the difference was again not statistically significant in the intestine due to greater variability between samples (FIGS. 4C and 4D).

### Pon3 Protein in Human Umbilical Cord Blood

A band of appropriate size for PON3 was detectable using Western blot of human cord serum. Cord serum levels of PON3 were compared for 8 preterm infants and 10 term infants. The mean gestational age of the preterm infants was 27 weeks and 3 days and the mean birth weight was 986 g. Four were normal deliveries and 4 were delivered by caesarean section. One infant had received a single dose of betamethasone prior to birth, and all the others had completed a course of antenatal steroids. Six out of eight infants had an initial CRP <5 mg/L and two had elevated initial CRP (14 mg/L and 68 mg/L). None had positive cultures of blood or CSF in the first sample performed following birth. PON3 levels were 6.3-fold higher at term (FIG. 8). When PON3 was expressed relative to total protein, assessed by Ponceau S staining, term levels were 4.9-fold higher and comparison of the log transformed means remained highly statistically significant (P<0.001).

Paraoxonase-3 was one of only 4 of the 28,532 genes on the array which was greater than 20-fold up-regulated in late gestation in both rat lung and intestines. This up-regulation was validated in siblings. Previous studies had also demonstrated that, in the human, PON3 was predominantly expressed in the liver²⁴ and we also observed up-regulation of Pon3 in this organ in late gestation in the rat. Western blot confirmed up-regulation at the protein level. We confirmed marked systemic up-regulation of PON3 mRNA in the last third of gestation in the fetal sheep, a phylogenetically distant mammalian species. Furthermore, like surfactant,¹⁹ expression of fetal sheep PON3 mRNA was controlled by glucocorticoids, as evidenced by strong correlations with endogenous cortisol levels, increased expression in preterm animals where dexamethasone had been given to the mother, and reduced expression at term among fetuses which had adrenalectomy performed at 115-118 dGA. PON3 circulates in the blood associated with low density lipoprotein.²⁵ Hence, we were able to study whether the protein is also up-regulated in the human infant in late gestation by measuring cord serum concentration. We found that PON3 levels were >6-fold higher among human infants born at term, despite the fact that 7 out of the 8 preterm infants studied had been exposed to a complete course of antenatal betamethasone. We conclude from these analyses that up-regulation of PON3 may be a systemic preparative process for birth which is observed in the rat, sheep and human fetus and is controlled by glucocorticoids.

The paraoxonase family consists of 3 proteins numbered 1-3. The genes encoding these proteins are located in a cluster on human chromosome 7q21-22 and share around 65% homology. All three are hydrolases and, specifically, hydrolyze a range of lactones and hydroxyl acids. Two studies have demonstrated that PON3 acts as an antioxidant. PON3, but not PON2, was found to protect low density lipoprotein (LDL) against copper-induced oxidation in vitro.²⁵ A recent study demonstrated that transgenic expression of human Pon3 in skeletal muscle of mice was associated with a reduced hepatotoxic effect of carbon tetrachloride (CCl₄).²⁶ The administration of CCl₄ causes liver damage through hepatic induction of free radicals. Transgenic human PON3 reduced levels of lipid peroxidation, and reduced biochemical and histological evidence of liver damage caused by CCl₄. Moreover, transgenic human Pon3 resulted in maintenance of normal hepatic levels of the endogenous anti-oxidants glutathione and superoxide dismutase, which were both depleted in control animals. Transgenic expression of human Pon3 in mice has also been shown to inhibit atherosclerotic lesion formation and adiposity.²⁷

The systemic nature of the up-regulation of PON3 in late gestation in the fetal rat suggests that it has some role which is required by different cell types in different organs. Moreover, the observation that Pon3 is systemically up-regulated in late gestation in both rats and sheep suggests that the up-regulation is related to a process common to diverse mammalian species around the time of birth. Following birth there is a rapid rise in the arterial partial pressure of oxygen in all mammalian species. In the sheep, the partial pressure of oxygen in fetal arterial blood is around 18-28 mmHg and this rises, within minutes following birth, to around 100 mmHg.²⁸ The rise in oxygen tension following birth is a key trigger for postnatal adaptation of the cardiovascular system, such as promoting closure of the ductus arteriosus^{16a} and increasing pulmonary blood flow.³⁰ However, rapid increases in oxygen tension can lead to the generation of reactive oxygen species (ROS) which can be defined as describing “all oxidation and excitation states of oxygen, from superoxide up to but excluding water, that arise in physiological environments”.³¹ Given the sudden increase in the partial pressure of oxygen following birth, it is likely that the neonate is exposed to increased generation of ROS following birth. Given the antioxidant effects of Pon3, the systemic up-regulation of the enzyme in late gestation may be conserved among diverse species due to a common requirement to limit oxidative stress immediately following birth.

Preterm birth is associated with increased morbidity and mortality. This is related to the fact that the baby is born prior to the normal preparative changes for neonatal life which occur in the fetus in late gestation. Surfactant therapy is an example of a successful clinical intervention which is based around supplementing preterm infants with a substance which would normally have been up-regulated in late gestation. These results show that administration of PON3 to the preterm human neonate is therefore likely to ameliorate the consequences of preterm birth and provide protection against complications of prematurity as described above.

A key property of PON3 is that it is found in the serum. Firstly, this allowed us to confirm up-regulation in human infants by measuring levels in serum obtained from umbilical cord blood samples. Secondly, if the role of PON3 in the neonate is mediated in part by increased circulating levels of the protein, this suggests that intravenous administration of exogenous Pon3 to the preterm neonate may be beneficial. This interpretation is supported by the studies of transgenic human PON3 in the mouse described above. Transgenic expression of Pon3 in skeletal muscle resulted in elevated Pon3 lactonase activity in the blood and this was associated with a reduced effect of CCl₄-induced free radical generation in the liver.²⁶ Hence, these observations are supportive of a systemic effect of circulating Pon3. Biochemical studies of the different paraoxonases have demonstrated that, although the three sub-types have some common substrates, they also are each able to hydrolyze specific substrates.³²

Three other genes were up-regulated more than 20-fold in late gestation in both the rat lung and intestine. Two of these were ion channels (solute carrier family 34, member 2 and chloride intracellular channel 5). Hence, neither is a potential candidate replacement therapy, although their systemic and profound up-regulation in late gestation provides indication that they are likely to be important in adaptation around the time of birth.

The remaining up-regulated transcript, serum amyloid A like protein 3, is an acute phase protein. Previous studies have addressed the effect of mechanical ventilation, endotoxin and glucocorticoids on expression of this protein in the fetal sheep.³³⁻³⁵ However, it is shown herein for the first time that physiological up-regulation at transcript level in the rat fetus in late gestation. Hence, serum amyloid A like protein 3 (SAA3) also represents a candidate novel therapeutic in the preterm neonate.

### REFERENCES

(1) Steer P. BJOG 2005; 112 Suppl 1:1-3.
(2) National Audit Office (UK). Caring for Vulnerable Babies: The reorganisation of neonatal services in England. London: The Stationery Office; 2007.
(3) Suresh G R, Soll R F. J Perinatol 2005; 25 Suppl 2:S40-S44.
(4) Heymann M A, Rudolph A M. Physiol Rev 1975; 55(1):62-78.
(5) Nathan C. J Clin Invest 2003; 111(6):769-778.
(6) Draganov D I. Circ Res 2007; 100(8):1104-1105.
(7) Crowley P. Cochrane Database Syst Rev 2000; (2):CD000065.
(8) Soll R F. Cochrane Database Syst Rev 2000; (2):CD001149.
(9) Forhead A J et al. Endocrinology 2002; 143(4):1166-1173.
(10) Draganov D I et al J. Lipid Res 2005; 46(6):1239-1247.
(11) Draganov D I et al J Biol Chem 2000; 275(43):33435-33442.
(12) Rudolph A M et al. Circ Res 1970; 26(3):289-299.
(13) Smith G C S. Pharmacol Rev 1998; 50(1):35-58.
(14) Clements J A. Annu Rev Physiol 1997; 59:1-21.
(15) Soll R F. Cochrane Database Syst Rev 2000; (2):CD001149.
(16) Brion L P, Bell E F et al Cochrane Database Syst Rev 2003; (3):CD003665.
(17) Suresh G K et al Cochrane Database Syst Rev 2001; (1):CD001968.
(18) Soghier L M et al Cochrane Database Syst Rev 2006; (4):CD004869.
(19) Tan R C et al Am J Physiol 1999; 277(6 Pt 1):L1142-L1148.
(20) Mostyn A et al. J Endocrinol 2003; 176(1):23-30.
(21) Long A D et al. J Biol Chem 2001; 276(23):19937-19944.
(22) Breitling R et al FEBS Lett 2004; 573(1-3):83-92.
(23) Vandesompele J et al Genome Biol 2002; 3(7):RESEARCH0034.
(24) Reddy S T et al Arterioscler Thromb Vasc Biol 2001; 21(4):542-547.
(25) Draganov D I et al. J Biol Chem 2000; 275(43):33435-33442.
(26) Peng W et al Biomed Pharmacother 2009; 63(8):592-598.
(27) Shih D M, et al. Circ Res 2007; 100(8):1200-1207.
(28) Heymann M A, Rudolph A M. Physiol Rev 1975; 55(1):62-78.
(29) Smith G C S. Pharmacol Rev 1998; 50(1):35-58.
(30) Lyrene R K et al Clin Perinatol 1984; 11(3):551-564.
(31) Nathan C. J Clin Invest 2003; 111(6):769-778.
(32) Draganov D I et al J Lipid Res 2005; 46(6):1239-1247.
(33) Hillman N H et al Am J Respir Crit Care Med 2007; 176(6):575-581.
(34) Kallapur S G et al. Am J Physiol Lung Cell Mol Physiol 2003; 284(4):L633-L642.
(35) Wilson T C at al Pediatr Res 2005; 58(6):1204-1209.
(36) Needleman et al J. Mol. Biol. (48): 444-453 (1970)

Sequences

| | |
|---|---|
| 1 | agtcgccgct gggcaccccg agaccatggg gaagctcgtg gcgctggtcc tgctgggggt |
| 61 | cggcctgtcc ttagtcgggg agatgttcct ggcgtttaga gaaagggtga atgcctctcg |
| 121 | agaagtggag ccagtagaac ctgaaaactg ccaccttatt gaggaacttg aaagtggctc |
| 181 | tgaagatatt gatatacttc ctagtgggct ggcttttatc tccagtggat taaaatatcc |
| 241 | aggcatgcca aactttgcgc cagatgaacc aggaaaaatc ttcttgatgg atctgaatga |
| 301 | acaaaaccca agggcacaag cgctagaaat cagtggtgga tttgacaaag aattatttaa |
| 361 | tccacatggg atcagtattt tcatcgacaa agacaatact gtgtatcttt atgttgtgaa |
| 421 | tcatccccac atgaagtcca ctgtggagat atttaaattt gaggaacaac aacgttctct |
| 481 | ggtatacctg aaaactataa aacatgaact tctcaaaagt gtgaatgaca ttgtggttct |
| 541 | tggaccagaa cagttctatg ccaccagaga ccactatttt accaactccc tcctgtcatt |
| 601 | ttttgagatg atcttggatc ttcgctggac ttatgttctt ttctacagcc caagggaggt |
| 661 | taaagtggtg gccaaaggat tttgtagtgc caatgggatc acagtctcag cagaccagaa |
| 721 | gtatgtctat gtagctgatg tagcagctaa gaacattcac ataatggaaa aacatgataa |
| 781 | ctgggattta actcaactga aggtgataca gttgggcacc ttagtggata acctgactgt |
| 841 | cgatcctgcc acaggagaca ttttggcagg atgccatcct aatcctatga agctactgaa |
| 901 | ctataaccct gaggaccctc caggatcaga agtacttcgc atccagaatg ttttgtctga |
| 961 | gaagcccagg gtgagcaccg tgtatgccaa caatggctct gtgcttcagg gcacctctgt |
| 1021 | ggcttctgtg taccatggga aaattctcat aggcaccgta tttcacaaaa ctctgtactg |
| 1081 | tgagctctag actctagata gtaaaaaaaa aaaaaaaaag tctacatatt ttgtaaaagt |
| 1141 | aaactgataa ttgtatgata agtggcactg taagtaaata gcaaacacca aaaaaaaaaa |
| 1201 | a |

(Genbank NM_000940.2 GI: 94538355)
SEQ ID NO: 1

| | |
|---|---|
| 1 | mgklvalvll gvglslvgem flafrervna srevepvepe nchlieeles gsedidilps |
| 61 | glafissglk ypgmpnfapd epgkiflmdl neqnpraqal eisggfdkel fnphgisifi |
| 121 | dkdntvylyv vnhphmkstv eifkfeeqqr slvylktikh ellksvndiv vlgpeqfyat |
| 181 | rdhyftnsll sffemildlr wtyvlfyspr evkvvakgfc sangitvsad qkyvyvadva |
| 241 | aknihimekh dnwdltqlkv iqlgtivdnl tvdpatgdil agchpnpmkl lnynpedppg |
| 301 | sevlriqnvl sekprvstvy anngsvlqgt svasvyhgki ligtvfhktl ycel |

(Genbank NP_000931.1 GI: 29788996)
SEQ ID NO: 2

| | |
|---|---|
| ATGGGGAAGCTCGTGGCGCTGGTCCTGCTGGGGGTCGGCCTGTCCTTAGTCGGGGAGATGTTTCTGGCGTTTAGAGAAAG | 80 |
| GGTGAATGCCTCTCGAGAAGTGGAGCCAGTAGAACCTGAAAACTGCCACCTTATTGAGGAACTTGAAAGTGGCTCTGAAG | 160 |
| ATATTGATATACTTCCTAGTGGGCTGGCTTTTATCTCCAGTGTCTGTTGGAGTTTGCTGGAAGTCCACTCCAGACCCTGT | 240 |
| TTGCCTGGGTATCACCAGTGGAGGCTGCAGAACGGCAAATATTGCTGCCTGATTTTTCTTCTGGAAGCTTCATCCCAGAG | 320 |
| GGGCATCCGCCTGTATGAGGGATTAAAATATCCAGGCATGCCAAACTTTGCGCCAGATGAACCAGGAAAAATCTTCTTGA | 400 |
| TGGATCTGAATGAACAAAACCCAAGGGCACAAGCACTAGAAATCAGTGGTGGATTTGACAAAGAATTATTTAATCCACAT | 480 |
| GGGATCAGTATTTTCATCGACAAAGACAATACTGTGTATCTTTATGTTGTGAATCATCCCCACATGAAGTCCACTGTGGA | 560 |
| GATATTTAAATTTGAGGAACAACAACGTTCTCTGGTATACCTGAAAACTATAAAACATGAACTTCTCAAAAGTGTGAATG | 640 |
| ACATTGTGGTTCTTGGACCAGAACAGTTCTATGCCACCAGAGACCACTATTTTACCAACTCCCTCCTGTCATTTTTTGAG | 720 |
| ATGATCTTGGATCTTCGCTGGACTTATGTTCTTTTCTACAGCCCAAGGGAGGTTAAAGTGGTGGCCAAAGGATTTTGTAG | 800 |
| TGCCAATGGGATCACAGTCTCAGCAGACCAGAAGTATGTCTATGTAGCTGATGTAGCAGCTAAGAACATTCACATAATGG | 880 |
| AAAAACATGATAACTGGGATTTAACTCAACTGAAGGTGATACAGTTGGGCACCTTAGTGGATAACCTGACTGTCGATCCT | 960 |
| GCCACAGGAGACATTTTGGCAGGATGCCATCCTAATCCTATGAAGCTACTGAACTATAACCCTGAGGACCCTCCAGGATC | 1040 |
| AGAAGTACTTCGCATCCAGAATGTTTTGTCTGAGAAGCCCAGGGTGAGCACCGTGTATGCCAACAATGGCTCTGTGCTTC | 1120 |
| AGGGCACCTCTGTGGCTTCTGTGTACCATGGGAAAATTCTCATAGGCACCGTATTTCACAAAACTCTGTACTGTGAGCTC | 1200 |
| TAG | 1203 |

PON3 long isoform nucleotide sequence (extra sequence shown in bold)
SEQ ID NO: 3

| | |
|---|---|
| MGKLVALVLLGVGLSLVGEMFLAFRERVNASREVEPVEPENCHLIEELESGSEDIDILPSGLAFISSVCWSLLEVHSRPC | 80 |
| LPGYHQWRLQNGKYCCLIFLLEASSQRGIRLYEGLKYPGMPNFAPDEPGKIFLMDLNEQNPRAQALEISGGFDKELFNPH | 160 |
| GISIFIDKDNTVYLYVVNHPHMKSTVEIFKFEEQQRSLVYLKTIKHELLKSVNDIVVLGPEQFYATRDHYFTNSLLSFFE | 240 |
| MILDLRWTYVLFYSPREVKVVAKGFCSANGITVSADQKYVYVADVAAKNIHIMEKHDNWDLTQLKVIQLGTLVDNLTVDP | 320 |
| ATGDILAGCHPNPMKLLNYNPEDPPGSEVLRIQNVLSEKPRVSTVYANNGSVLQGTSVASVYHGKILIGTVFHKTLYCEL | 400 |

PON3 long isoform amino acid sequence (extra sequence shown in bold)
SEQ ID NO: 4

| | |
|---|---|
| 1 | aacttgaaac agaatgtgta ttatccttgg ttgtgtttcc ttggccctgc agcaggatga |
| 61 | agctctcctc tggcatcatt ttctgctccc tggtcctggg tgtcagcagc caaggatggt |
| 121 | taacattcct caaggcagct ggccaaggga ctaaagacat gtggaaagcc tactctgaca |
| 181 | tgaaagaagc caattacaaa aaattcagac aaatacttcc atgcttgggg gaactatgat |
| 241 | gctgtacaaa gggggcttgg ggctgtctgg gctacagaag tgatcaggta atgcacattc |
| 301 | ctgatgttgc caggaatgag tgagcagagc ttgactgcct tggacagtca ggagagagcg |
| 361 | atgccagaga gaacgtccag agactcacag gagaccatgc agaggattcg ctggctggcc |
| 421 | aggctaccaa caaatggggc cagagtggca aagaccccaa tcacttccga cctgctggcc |
| 481 | tgccagagaa atactgagct tccttttcaa tctgctctca ggagacctgg ctgtgagccc |
| 541 | ctgagggcag ggacatttgt tgacctacag ttactgaatt ctatatccct agtacttgat |
| 601 | atagaacaca taaaaatgct taataaatgc ttgtgaaatc caaaaaaaaa aaaaa |

human SAA3 nucleotide sequence (AY209188.1 GI: 28864697)
SEQ ID NO: 5

| | |
|---|---|
| 1 | mklssgiifc slvlgvssqg wltflkaagq gtkdmwkays dmkeanykkf rqilpclgel |

human SAA3 amino acid sequence (AAO48437.1 GI: 28864698)
SEQ ID NO: 6

| | | | |
|---|---|---|---|
| Ovine | Pon3 | CCCTAGTCGGGGAGAGATTTTTGACATTTAGAGAAAGAATGAATGCATATCGRGAAGTGG | 60 |
| Bovine | Pon3 | CCCTAGTCGGGGAGAGATTTTTGACACTTCGAGAAAGAATGAATGCATATCGAGAAGTGG | 60 |
| Human | Pon3 | CCTTAGTCGGGGAGATGTTCCTGGCGTTTAGAGAAAGGGTGAATGCCTCTCGAGAAGTGG | 60 |
| | | ** ************ ** ** * ** ******* ******* * *** ******* | |
| Ovine | Pon3 | AGTCAGTAGAACCCCAGAACTGCCACCTTATTGAGGGAATTGAAAATGGCTCTGAAGATA | 120 |
| Bovine | Pon3 | AGTCAGTAGAATCCCAGAACTGCCACCTTATTGAGGGAATTGAAAATGGCTCTGAAGATA | 120 |
| Human | Pon3 | AGCCAGTAGAACCTGAAAACTGCCACCTTATTGAGGAACTTGAAAGTGGCTCTGAAGATA | 120 |
| | | ** ******** * * ******************* * ****** ************** | |
| Ovine | Pon3 | TTGATATCCTTCCTAGTGGGCTGGCTTTCATCTCCAGTGGATTAAAATATCCAGGCATGC | 180 |
| Bovine | Pon3 | TTGATATCCTTCCTAGTGGGCTGGCTTTCATCTCCAATGGATTAAAATATCCAGGCATGC | 180 |
| Human | Pon3 | TTGATATACTTCCTAGTGGGCTGGCTTTTATCTCCAGTGGATTAAAATATCCAGGCATGC | 180 |
| | | ******* ******************** ******* *********************** | |
| Ovine | Pon3 | CAGACTTTGCACCAGATRAGCCAGGACGAATCTTCTTGATGGATTTAAATGAGCAAAACC | 240 |
| Bovine | Pon3 | CAGACCTTGCACCAGATGAGCCAGGACGAATCTTCTTGATGGATTTAAATGAGCAAAACC | 240 |
| Human | Pon3 | CAAACTTTGCGCCAGATGAACCAGGAAAAATCTTCTTGATGGATCTGAATGAACAAAACC | 240 |
| | | ** ** **** ****** * ****** **************** * ***** ******* | |
| Ovine | Pon3 | CTAGGGTCCAGGAATTAAACATCAGTGATGGTTTTGACAAAGCATCCTTTAATCCACATG | 300 |
| Bovine | Pon3 | CTAGGGTCCAGGAATTAAACATCAGTGATGGTTTTGACAAAGCATCCTTTAATCCACATG | 300 |
| Human | Pon3 | CAAGGGCACAAGCGCTAGAAATCAGTGGTGGATTTGACAAAGAATTATTTAATCCACATG | 300 |
| | | * **** ** * ** * ******* *** ********** ** ************* | |
| Ovine | Pon3 | GAATGAGTACTTYCATTGACAAAGACCAAACTGTGTATCTTTATGTTGTGAATCATCCCC | 360 |
| Bovine | Pon3 | GAATGAGTACTTTCATTGACAAAGACCAAACTGTATATCTTTATGTTGTGAATCATCCCC | 360 |
| Human | Pon3 | GGATCAGTATTTTCATCGACAAAGACAATACTGTGTATCTTTATGTTGTGAATCATCCCC | 360 |
| | | * ** **** ** *** ********* * ***** ************************* | |
| Ovine | Pon3 | ACATGGRCTCCACCGTGGAGATATTTAAATTTGAGGAACAACAACGTTCTTTRGTATACC | 420 |
| Bovine | Pon3 | ACATGGACTCCACCGTGGAGATATTTAAATTTGAAGAACAACAACGTTCTTTGGTATACC | 420 |
| Human | Pon3 | ACATGAAGTCCACTGTGGAGATATTTAAATTTGAGGAACAACAACGTTCTCTGGTATACC | 420 |
| | | ***** ***** ******************** *************** * ******* | |
| Ovine | Pon3 | TGAAAACTATAAAACATGAACTTCTCAAAAGTGTGAATGATATCGTGGTTCTCGGACCAG | 480 |
| Bovine | Pon3 | TGAAAACTATAAAACACGAACTTCTCAAAAGTGTGAATGATATTGTGGTTCTCGGACCAG | 480 |
| Human | Pon3 | TGAAAACTATAAAACATGAACTTCTCAAAAGTGTGAATGACATTGTGGTTCTTGGACCAG | 480 |
| | | **************** *********************** ** ******** ******* | |
| Ovine | Pon3 | AAGAGTTCTATGCCACCAGNGACCA | 505 |
| Bovine | Pon3 | AACAGTTCTATGCCACCAGAGACCA | 505 |
| Human | Pon3 | AACAGTTCTATGCCACCAGAGACCA | 505 |
| | | ** **************** ***** | |

Alignment of the partial sheep PON3 cDNA sequence with the homologous sequences of bovine and human PON3.

Ambiguous bases that can indicate sequencing errors or single nucleotide polymorphisms in the sheep sequence are marked as follows: R=A/G, Y=C/T. * denotes homology between all three species for the given nucleotide. Accession numbers: human=NM_{—}000940.2; bovine=NM_{—}001075479.1; and ovine=GU327780.

### Tables

TABLE 1

| Gene | Forward primer | Reverse primer | Probe |
|---|---|---|---|
| Pon3 | CCTAGTGGGCTGGCTTTCATC | CTGGCTTATCTGGTGCAAAGTCT | TCCAGTGGATTAAAATATC |
| Gapdh | GCTACACTGAGGACCAGGTT | AGCATCGAAGGTAGAAGAGTGAGT | CTCCTGCGACTTCAAC |
| Cyclophilina | GGTTCCTGCTTTCACAGAATAATTCC | GTACCATTATGGCGTGTGAAGTCA | CACCCTGGCACATAAA |
| β-Actin | CCGTCTTCCCTTCCATCGT | CCCACGTAGGAGTCCTTCTG | CATCACGCCCTGGTGCC |

TABLE 2

| Gene | Lungs | Intestines |
|---|---|---|
| >20-fold both | | |
| tissues | | |
| Saa3 | 38* | 24* |
| Pon3 | 24* | 31* |
| Slc34a2 | 215* | 48* |
| Clic5 | 48* | 63* |
| Other Pon | | |
| Pon1, probe 1 | 1.5 | 1.6 |
| Pon1, probe 2 | −1.6 | 1.2 |
| Pon2 | 2.7 | 1.9 |

Abbreviations:
Saa3 = serum amyloid A like protein 3;
Pon = paraoxonase;
Slc34a2 = solute carrier family 34, member 2;
Clic5 = chloride intracellular channel 5.
n = 10 at both 16dGA and 20dGA, with all samples in a given tissue at a given gestational age taken from a single animal from a given litter.
*Statistically significantly fold change between 16dGA and 20dGA using both Cyber-T (Bayes P value <0.001, posterior probability of differential expression >0.99) and Rank Product Analysis (P < 0.0001).

1. A method of treatment of a pre-term neonate comprising:
administering paraoxonase 3 (PON3) polypeptide to the pre-term neonate.

2. A method according to claim 1 wherein the treatment reduces the risk of mortality and/or the extent, severity or risk of morbidity in said neonate

3. A method according to claim 1 wherein the PON3 polypeptide has at least 80% sequence identity to SEQ ID NO: 2 or SEQ ID NO:4.

4. A method according to claim 1 wherein the PON3 polypeptide is formulated in a pharmaceutical composition.

5. A method according to claim 1 wherein the PON3 polypeptide is administered parenterally.

6. A method according to claim 5 wherein the PON3 polypeptide is administered intravenously.

7. A method according to claim 1 wherein the PON3 polypeptide is administered enterally.

8-19. (canceled)

20. A method according to claim 1 comprising identifying the pre-term neonate as deficient in PON3 before said administration.

21. A method of identifying a pre-term neonate at risk of morbidity and/or mortality comprising:
determining the level or activity of PON3 in a sample of obtained from the neonate;
wherein a reduced level or activity of PON3 relative to controls is indicative that the pre-term neonate is at risk of morbidity and/or mortality.
